(19) United States
(12) Patent Application Publication
(10) Pub. No.: US 2024/0306999 A1
(43) Pub. Date: Sep. 19, 2024

(54) METHOD FOR CALIBRATING A MOVEMENT DETECTION METHOD AND A MAGNETIC RESONANCE APPARATUS

(71) Applicant: Siemens Healthineers AG, Forchheim (DE)

(72) Inventors: Julian Wohlers, Erlangen (DE); Daniel Nicolas Splitthoff, Uttenreuth (DE); Tobias Kober, Lausanne (CH); Hans Weber, Erlangen (DE)

(21) Appl. No.: 18/602,387

(22) Filed: Mar. 12, 2024

(30) Foreign Application Priority Data
Mar. 15, 2023 (DE) 10 2023 202 343.1

(51) Int. Cl. A61B 5/00; A61B 5/055; G01R 33/58; G01S 7/40

(52) U.S. Cl. CPC A61B 5/721; A61B 5/0042; A61B 5/055; A61B 5/7267; G01R 33/58; G01S 7/40; A61B 2560/0223

(57) ABSTRACT

A movement detection method is calibrated for a magnetic resonance apparatus. An examination object is positioned on a movement apparatus. During a training phase, training data is recorded according to the movement detection method, wherein the examination object is simultaneously moved by the movement apparatus. The movement detection method is calibrated on the basis of the training data.

### RELATED APPLICATION

This application claims the benefit of DE 10 2023 202 343.1, filed on Mar. 15, 2023, which is hereby incorporated by reference in its entirety.

### FIELD

The present disclosure relates to calibrating a movement detection method and to a magnetic resonance apparatus.

### BACKGROUND

In medical engineering, imaging by magnetic resonance (MR), also called Magnetic Resonance Tomography (also Magnetic Resonance Imaging, MRI), is characterized by high soft tissue contrasts. Here, an examination object, for example a patient, is typically positioned in a static, homogeneous magnetic field of a magnetic resonance apparatus. During a magnetic resonance measurement, radio frequency (RF) transmission pulses are conventionally irradiated into the examination object in accordance with a magnetic resonance sequence. Nuclear spins are excited in the examination object by the generated transmission pulses in connection with the static magnetic field, whereby spatially encoded magnetic resonance signals are triggered by gradient pulses. The magnetic resonance signals are received from the magnetic resonance apparatus and are used for the reconstruction of MR mappings.

Documents EP1489966A1, U.S. Pat. Nos. 5,772,595A, 5,743,264A, 5,541,515A, 5,899,859A, 5,154,178A, 5,724,970A, 5,520,181A, 5,445,152A, 5,442,858A and 4,979,519A disclose movement apparatuses for the movement of a body part of a patient in a magnetic resonance apparatus in order to thereby carry out, in particular, kinematic studies.

Various movement detection methods are known which are suitable for capturing a movement of an examination object during a magnetic resonance measurement. For example, the magnetic resonance measurement can be controlled and/or a prospective and/or retrospective movement correction can be carried out on the basis of the detected movement.

A movement detection method can advantageously be improved by training it with the movement to be detected. The movement detection method is consequently capable of identifying the movement to be detected more purposefully and/or more robustly. The object is to improve recording of training data for a movement detection method.

### SUMMARY AND DETAILED DESCRIPTION

The object is achieved by the features described below. Advantageous embodiments are described.

Independent of the grammatical term usage, individuals with male, female or other gender identities are included within the term.

A method for calibrating a movement detection method is being proposed. The movement detection method is suitable for capturing a movement of an examination object during a magnetic resonance measurement. The method includes positioning of the examination object on a movement apparatus and recording the training data in accordance with the movement detection method in a training phase. During the training phase, the examination object is moved by the movement apparatus. The movement detection method is calibrated on the basis of the training data.

Preferably, the movement detection method provides a recording of signals, on the basis of which a, in particular physiological, movement of the examination object can be detected. Such signals can be, for example, pilot signals modulated, in particular, by a movement of the examination object. Further possible movement detection methods can provide that such signals are MR navigator signals, noise correlation signals, magnetic field signals captured, in particular, by a pick-up coil, laser signals, radar signals, in particular shortwave radar signals, and/or, optical signals captured, in particular, by camera. Preferably, such signals are recorded as training data in the training phase.

The training phase can be part of a magnetic resonance examination. Such a magnetic resonance examination conventionally includes a measuring period in which a magnetic resonance measurement is carried out. Magnetic resonance signals are conventionally recorded during the measuring period, on the basis of which signals (diagnostic) magnetic resonance mappings can be reconstructed. The training data can be recorded, in particular, before, during, and/or after the measuring period.

The examination object (to be moved) can be, in particular, a patient, in particular a body part of the patient, in particular the head of the patient.

Training of the movement detection method on the basis of the training data can follow recording of the training data. The movement detection method trained as a result can advantageously capture the movement to be detected during a magnetic resonance measurement more purposefully and/or more robustly. Such a magnetic resonance measurement can then be carried out, for example after training of the movement detection method on the basis of the training data.

The use of a movement apparatus means the training movement of the examination object can be purposefully carried out. It is thus not necessary, in particular, to prompt the patient to actively carry out such training movements. Instead, the movement of the examination object by the movement apparatus is a passive movement of the examination object. Generation of a training movement by a movement apparatus is advantageous, in particular, in the case of movements that are involuntarily carried out by the patient and/or are not spontaneously carried out in any case, such as cardiac or respiratory movement. Thus, in particular, the workflow of a magnetic resonance examination can be simplified, in particular be automated (to a greater extent).

Preferably, the movement of the examination object by the movement apparatus has a periodic oscillation, in particular a vibration. A movement of this kind is particularly suitable for training the movement detection method.

Preferably, the oscillation has a frequency which is less than 20 Hz, in particular less than 10 Hz, and/or greater than 0.001 Hz, in particular greater than 0.01 Hz, in particular greater than 0.1 Hz. The periodic oscillation thereby advantageously lies in the infrasonic spectrum, which is inaudible to humans. The movement apparatus will advantageously cause the examination object, for example the head of a patient, to move slightly and/or subtly.

Preferably, the movement detection method includes an interaction of a pilot signal with the moving examination object. Preferably, the movement detection method is a pilot signal method. In this case, a pilot signal is emitted by a pilot signal generator, which signal is modulated by a movement of the examination object and is recorded, in particular captured, by the magnetic resonance apparatus. In particular, modulated pilot signals are recorded as training data in the training phase. Advantageously, the pilot signals are modulated during the training phase by the movement of the examination object by the movement apparatus.

Advantageously, the magnetic resonance apparatus has a receiving bandwidth large enough to simultaneously receive a magnetic resonance signal and a pilot signal, which does not lie in the frequency range of the magnetic resonance signal. The magnetic resonance apparatus can include a plurality of receiving elements, in particular coil elements, which are each assigned to a receiving channel. Reference should be made to documents US 20160245888 A1, US20170,160364 A1 and US 20180353139 A1 for further aspects of the pilot signal method.

Furthermore, a magnetic resonance apparatus with a movement apparatus for recording calibration data for a movement detection method according to a previously described method is being proposed.

The advantages of the proposed magnetic resonance apparatus substantially correspond to the advantages of the proposed method for calibrating a movement detection method, which advantages have been described previously in detail. Features, advantages, or alternative embodiments mentioned in this connection can be transferred to the proposed magnetic resonance apparatus, and vice versa.

Preferably, the movement apparatus is part of a head coil. A head coil is conventionally a specific local coil in which the head of a patient can be positioned during the magnetic resonance measurement. The head coil is embodied to receive magnetic resonance signals. For this, the head coil can include a plurality of coil elements, in particular RF antennas, in which the magnetic resonance signals can be induced. Furthermore, the head coil can be embodied to transmit RF transmission signals, in particular in order to excite nuclear spins in the examination object. The head coil can include, for example, an upper part and a lower part, wherein the upper part can advantageously be removed for improved positioning of the head.

The movement apparatus is arranged, for example, in a lower region of the head coil on which the head of the patient is supported. Preferably, after positioning of the examination object on the movement apparatus, the movement apparatus is in direct contact with the head of the patient, so the movement can be transferred particularly effectively from the movement apparatus to the head.

Preferably, the movement apparatus includes at least one actuator, which generates the movement. The at least one actuator is preferably embodied to convert movement control data, in particular an electrical signal, into a mechanical movement. For example, the actuator includes at least one piezo element. The at least one piezo element is preferably embodied to execute a mechanical movement by the piezo effect when an electrical voltage is applied. Piezo elements are advantageously compact, so they may be easily integrated in a head coil. Furthermore, piezo elements advantageously have high MR compatibility.

Preferably, the magnetic resonance apparatus includes a calibration unit which is embodied to receive the training data and calibrate the movement detection method on the basis of the training data. The calibration unit can include, in particular, one or more processor(s) and/or one or more storage module(s) (memories) for processing the training data.

Preferably, the magnetic resonance apparatus includes a movement control unit (controller), which is embodied to control the movement on the basis of movement control data, which movement is executed by the movement apparatus.

For example, the movement control unit provides a voltage by way of which at least one piezo element of the movement apparatus is actuated. Advantageously, the movement of the examination object can be purposefully carried out in the training phase by the movement control unit.

Preferably, the calibration unit is embodied to receive the movement control data and to calibrate the movement detection method on the basis of the movement control data. If a, in particular mathematical, relationship is established between the movement control data and the training data, the calibration can occur particularly precisely.

### BRIEF DESCRIPTION OF THE DRAWINGS

Further advantages, features, and details of the invention result from the exemplary embodiments described below and on the basis of the drawings. Mutually corresponding parts are provided with identical reference numerals in all figures.

In the drawings:

FIG. 1 shows an embodiment of a magnetic resonance apparatus with a head coil in which a movement apparatus is integrated,

FIG. 2 shows an embodiment of a head coil with the movement apparatus in detail,

FIG. 3 shows a diagram of one embodiment of a method for calibrating a movement detection method.

### DETAILED DESCRIPTION

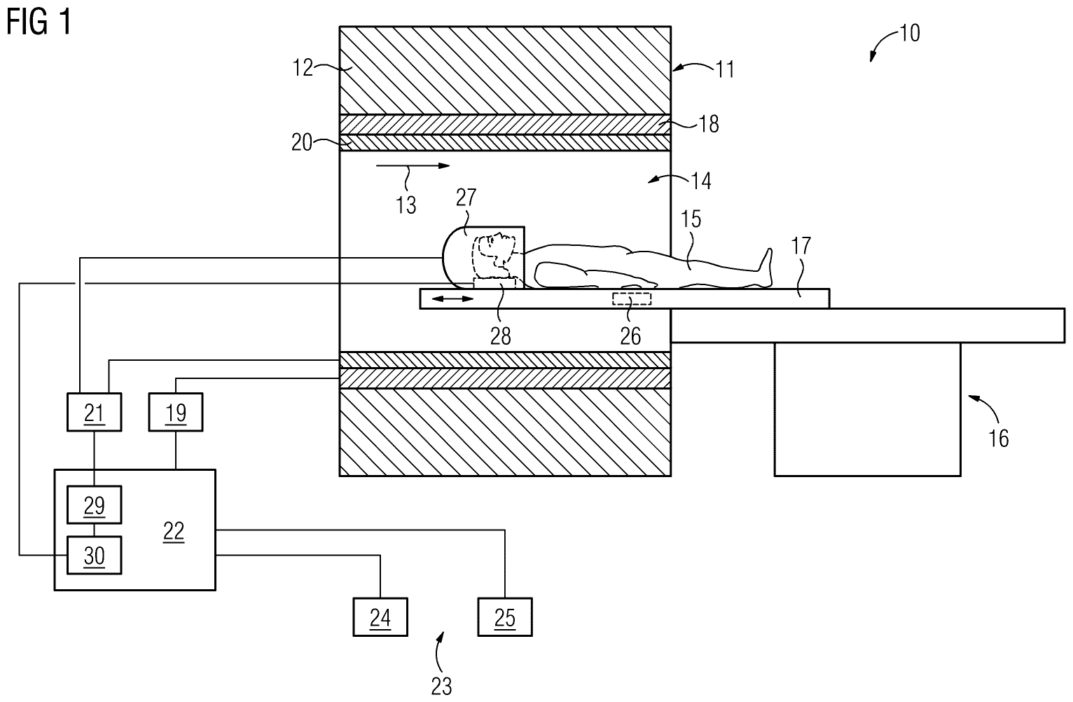

FIG. 1 schematically represents a magnetic resonance apparatus 10. The magnetic resonance apparatus 10 includes a magnetic unit 11 which has a main magnet 12 for generating a strong and, in particular, temporally constant main magnetic field 13. In addition, the magnetic resonance apparatus 10 includes a patient-receiving region 14 for receiving a patient 15 as an examination object. The patient-receiving region 14 in the present exemplary embodiment is cylindrical and cylindrically surrounded by the magnetic unit 11 in a circumferential direction. In principle, a different design of the patient-receiving region 14 is conceivable, however. The patient 15 can be pushed into the patient-receiving region 14 by a patient-supporting apparatus 16 of the magnetic resonance apparatus 10. The patient-supporting apparatus 16 has for this purpose a patient table 17 configured to move inside the patient-receiving region 14.

The magnetic unit 11 also has a gradient coil or gradient coil unit 18 for generating magnetic field gradients used for spatial encoding during an imaging process. The gradient coil unit 18 is controlled by a gradient control unit 19 (controller) of the magnetic resonance apparatus 10. The magnetic unit 11 also includes a radio frequency (RF) antenna or RF antenna unit 20, which is embodied in the present exemplary embodiment as a body coil permanently integrated in the magnetic resonance apparatus 10. The radio frequency antenna unit 20 is controlled by a radio frequency antenna control unit 21 (controller) of the magnetic resonance apparatus 10 and irradiates radio frequency excitation pulses into an examination space formed substantially by a patient-receiving region 14 of the magnetic resonance apparatus 10. The main magnetic field 13 generated by the main magnet 12 consequently establishes an excitation of atomic nuclei. Magnetic resonance signals are generated by relaxation of the excited atomic nuclei. The radio frequency antenna unit 20 is embodied to receive the magnetic resonance signals and is part of an RF receiving unit (receiver) of the magnetic resonance apparatus.

The magnetic resonance apparatus 10 has a system control unit 22 (controller) for controlling the main magnet 12, the gradient control unit 19, and the radio frequency antenna control unit 21. The system control unit 22 centrally controls the magnetic resonance apparatus 10, such as, in particular, carrying out a magnetic resonance sequence. In addition, the system control unit 22 includes an evaluation unit (not represented) for evaluating the magnetic resonance signals captured during the magnetic resonance examination. Furthermore, the magnetic resonance apparatus 10 includes a user interface 23 connected to the system control unit 22. Control information, such as imaging parameters, and reconstructed magnetic resonance mappings, can be displayed on a display unit 24, for example on at least one monitor, of the user interface 23 for a medical operator. Furthermore, the user interface 23 has an input or input unit 25, by which items of information and/or parameters can be input by the medical operator during a measuring procedure.

Furthermore, the magnetic resonance apparatus 10 includes, in particular as part of the radio frequency antenna unit 20, a local coil in the form of a head coil 27 in which the head of the patient 15 is arranged. The head coil 27 is embodied for receiving the magnetic resonance signals. It is also conceivable that the head coil 27 is embodied for transmitting radio frequency excitation pulses. The head coil 27 includes a movement apparatus 28 for moving the head of the patient.

In order to carry out a movement detection method to be calibrated, here: the pilot signal method, the magnetic resonance apparatus also includes a pilot signal generator 26 arranged here in the patient table 17. It is also conceivable, however, that the pilot signal generator 26 is arranged at a different location of the magnetic resonance apparatus 10, such as in a local coil, such as the head coil 27 depicted here. The pilot signal method is an electromagnetic, contactless movement detection method. According to this method, the pilot signal generator 26 emits a pilot signal which is modulated by a movement of the patient 15 and is captured by the radio frequency antenna unit 20 of the magnetic resonance apparatus 10, in particular by the head coil 27. Such signals captured during a training phase can be training data for calibrating the pilot signal method. A movement of the patient 15 can be derived from these modulated signals. The pilot signal method therefore includes an interaction of the pilot signal with the moving patient 15. The pilot signal generator 26 can be controlled, for example, by the system control unit 22. Advantageously, the radio frequency antenna unit 20, in particular the head coil 27, has a receiving bandwidth large enough to simultaneously receive the magnetic resonance signal and pilot signal, which advantageously does not lie in the frequency range of the magnetic resonance signal. The head coil 27 can include a plurality of coil elements which are each assigned to a receiving channel.

The system control unit 22 has a calibration unit 29 (processor), which is embodied to receive the training data, in particular the pilot signals captured during the training phase. Furthermore, the system control unit 22 has a movement control unit 30 (controller), which is embodied to control the movement on the basis of movement control data, which movement is executed by the movement apparatus 28. Preferably, the calibration unit 29 is embodied to receive the movement control data from the movement control unit 30 in order to take it into account during calibration of the pilot signal method.

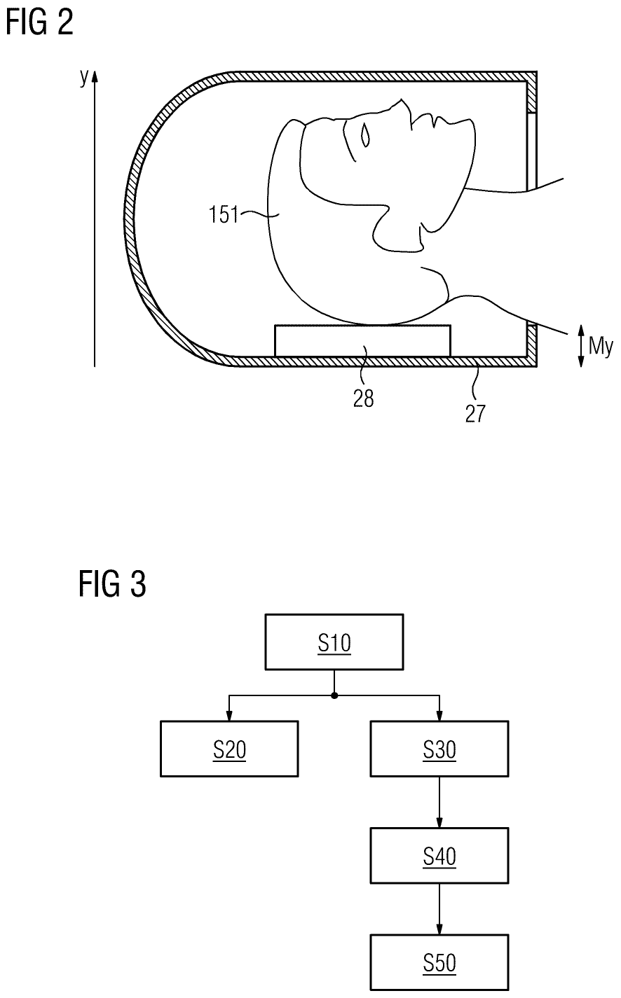

FIG. 2 represents the head coil 27 with the movement apparatus 28 once again in detail. The head 151 of the patient 15 is positioned on the movement apparatus 28. In this example, the movement apparatus 28 is embodied to carry out a movement, My, in the y-direction. For this, the movement apparatus 28 can include, for example, an actuator in the form of a piezo element which expands or contracts in the y-direction when a voltage is applied. The application of the voltage can be controlled, for example, by the movement control unit 30. The head 151 lying on the movement apparatus 28 also moves due to the movement of the piezo element.

The movement of the movement apparatus 28 can be executed, in particular, as a periodic oscillation in that the movement apparatus 28 vibrates. Such a vibration can lie, in particular, in the spectral range of infrasound, so the patient 15 does not experience any noise disturbance during the training phase.

FIG. 3 represents a possible development of a method for calibrating the pilot signal method. In S10, the head 151 of the patient 15 is positioned on the movement apparatus 28. During a training phase, on the one hand, the head 151 of the patient 15 is moved in S20 by the movement apparatus 28 and on the other hand, training data (in the form of pilot signals modulated by the movement of the head 151) is recorded in S30 in accordance with the pilot signal method. During a training phase, the training data is therefore simultaneously recorded in accordance with the pilot signal method while the examination object is being moved by the movement apparatus 28.

The pilot signal method can be calibrated in S40 on the basis of the training data. The actual magnetic resonance measurement can occur in S50 during which, in particular, the movement of the head 151 is captured by the now calibrated pilot signal method.

By the movement of the head 151 forced by the movement apparatus 28, it is not necessary to instruct the patient 15 to independently move their head 151 during the training phase. The workflow of a magnetic resonance examination can thus be improved, in particular automated.

In conclusion, reference will be made once again to the fact that the method described above in detail and the represented magnetic resonance apparatus are merely exemplary embodiments which can be modified in a wide variety of ways by a person skilled in the art without departing from the scope of the invention. Furthermore, use of the indefinite article “a” or “an” does not preclude the relevant features from also being present several times. Similarly, the term “unit” does not preclude the relevant components from consisting of a plurality of cooperating sub-components which can optionally also be spatially distributed.

1. A method for calibrating a movement detection method, the method comprising:
positioning an examination object on a movement apparatus,
recording training data according to the movement detection method in a training phase with movement of the examination object by the movement apparatus during the training phase, and
calibrating the movement detection method on the basis of the training data.

2. The method as claimed in claim 1, wherein the movement of the examination object by the movement apparatus has a periodic oscillation.

3. The method as claimed in claim 2, wherein the periodic oscillation has a frequency less than 20 Hz.

4. The method as claimed in claim 3, wherein the periodic oscillation has a frequency greater than 0.001 Hz.

5. The method as claimed in claim 1, wherein the examination object is a head of a patient.

6. The method as claimed in claim 1, wherein the movement detection method comprises an interaction of a pilot signal with the moving examination object.

7. The method as claimed in claim 1, wherein recording the training data comprises recording signals, wherein the signals comprise a pilot signal, modulated by a movement of the examination object, a magnetic resonance navigator signal, a noise correlation signal, a magnetic field signal captured by a pick-up coil, a laser signal, a radar signal, and/or an optical signal captured by a camera.

8. The method as claimed in claim 3, wherein the frequency is less than 10 Hz and greater than 0.1 Hz.

9. The method as claimed in claim 7 wherein the signals comprise the radar signal, the radar signal comprising a shortwave radar signal.

10. A magnetic resonance apparatus comprising:
a movement apparatus configured to move an examination object,
a sensor configured to recording calibration data for a movement detection method based on the movement of the examination object by the movement apparatus, and a processor configured to calibrate the movement detection method in a training phase with the recorded calibration data.

11. The magnetic resonance apparatus as claimed in claim 10, wherein the movement apparatus is part of a head coil.

12. The magnetic resonance apparatus as claimed in claim 10, wherein the movement apparatus comprises at least one actuator which generates the movement.

13. The magnetic resonance apparatus as claimed in claim 12, wherein the actuator comprises at least one piezo element.

14. The magnetic resonance apparatus as claimed in claim 10, wherein the processor comprises a calibration unit configured to receive the calibration data as training data, wherein the calibration unit is configured to calibrate the movement detection method on the basis of the training data.

15. The magnetic resonance apparatus as claimed in claim 10, further comprising a movement controller configured to control the movement on the basis of movement control data, which movement is executed by the movement apparatus.

16. The magnetic resonance apparatus as claimed in claim 15, wherein the processor is configured to receive the movement control data and to calibrate the movement detection method on the basis of the movement control data.
